(19) United States
(12) Patent Application Publication
(10) Pub. No.: US 2016/0263167 A1
(43) Pub. Date: Sep. 15, 2016

(54) PHYTOCHEMICAL COMPOSITIONS AND TECHNIQUES FOR IMMUNOMODULATION, INCLUDING ANTI-PATHOGEN AND ANTI-AUTOIMMUNE-DISORDER IMMUNOMODULATION

(71) Applicant: ASIAN PHYTOCEUTICALS PUBLIC COMPANY LIMITED, Muang, Lamphun (TH)

(72) Inventor: Pichaet WIRIYACHITRA, Bangkok (TH)

(21) Appl. No.: 15/033,504

(22) PCT Filed: Oct. 30, 2013
(86) PCT No.: PCT/TH2013/000060

§ 371 Date: Apr. 29, 2016

(51) Int. Cl. A61K 36/185; A61K 36/23; A61K 36/48; A61K 36/38; A61K 31/352

(52) U.S. Cl. CPC A61K 36/185; A61K 36/38; A61K 31/352; A61K 36/48; A61K 36/23

(57) ABSTRACT

A phytochemical anti-pathogen/anti-cancer composition is provided that includes an extract of mangosteen; an extract of defatted soybean; an extract of gotu kola; and optionally an extract of black sesame, where an amount of the extract of black sesame is controlled relative to an amount of the extract of gotu kola, or an amount of a combination of the extract of rriangosteen, the extract of defatted soybean, and the extract of gotu kola, in order to preferentially support antipathogen/anti-cancer immunomodulatoty effects. A phytochemical anti-autoimmune disorder composition containing the same four ingredients, where an amount of the extract of gotu kola is controlled relative to the amounts of the other extracts is also provided. The two compositions can support or provide antipode immunomodulatory effects relative to each other.

### TECHNICAL FIELD

The present disclosure relates generally to compositions capable of exhibiting immunomodulatory effects within a subject such as a human being or an animal. More particularly, aspects of the present disclosure are directed to a first composition having a first set of components can support or provide anti-pathogen/anti-cancer immunomodulatory effects, and a second composition having a second set of components can support or provide anti-autoimmune-disorder immunomodulatory effects. The first and second compositions can be separately or sequentially administrable to and/or absorbable within a subject in a manner that supports or provides antipode immunomodulatory effects or responses.

### BACKGROUND

The immune system of a subject such as a human being or an animal provides the foundation for fighting pathogens, such as viruses, bacteria, and fungi, which have invaded or which have been introduced into the subject's body. The immune system's recognition of pathogens triggers an immune response in the body, which leads to increased production of particular pro-inflammatory mediators or pro-inflammatory cytokines within the body.

More specifically, white blood cells (WBCs), or leukocytes, are a part of immune system, particularly the adaptive immune system. White blood cells are essential for fighting pathogens, for example, bacteria, viruses, and other foreign matters that invade the body of living organisms causing infection and diseases. Elevated WBCs are a sign of an infection. In a normal adult body there are approximately 4,000 to 10,000 (average 7,000) WBCs per microliter of blood or approximately 20,000-55,000 million white blood cells in a healthy male. White blood cells can be categorized into three groups according to their functions, namely: 1) phagocyte, NK (natural killer) cells and cytotoxic T-cells; 2) B-cell and plasma cells; and, 3) Tₕ or T-helper cells (which stimulate immune reactive processes). The amount of each type of white blood cell within the body must be properly regulated to maintain bodily homeostasis.

Achieving an enhanced immune system response to pathogen attack or the presence of cancer cells without increasing the likelihood of inducing an auto immune type reaction can be problematic. For instance, when a given particular drug, pharmaceutical composition, or substance is introduced to a body of a living organism in order to stimulate the organism's immune system, such a drug, composition, or substance can effectuate an increase in number of one type of white blood cells to boost the immune system (e.g, Tₕ-1), but this can concurrently lead to excess or imbalance in number of another type of immune cell (e.g. Tₕ-2). Such an imbalance can manifest in, for example, an autoimmune disease (where body exhibits elevated WBC levels and attacks itself) or leucopenia (where the body exhibits reduced levels of WBC making it vulnerable to infection).

There is a need for new and/or enhanced compositions and techniques for modulating a subject's immune system, particularly in a manner that avoids giving rise to chronic immune system imbalances, and which can facilitate the establishment or restoration of immune system balance.

### SUMMARY

In accordance with an aspect of the present disclosure, a phytochemical composition for facilitating or providing immunomodulatory effects in a subject includes an extract of mangosteen providing both alpha mangostin and gamma mangostin; an extract of defatted soybean; an extract of gotu kola; and an extract of black sesame, wherein an amount of the extract of black sesame is less than or equal to approximately 45% of the amount of an amount of a combination of the extract of mangosteen, the extract of defatted soybean, and the extract of gotu kola such that the immunomodulatory effects facilitated or provided by the composition are preferentially or predominantly anti-pathogen/anti-cancer immunomodulatory effects (e.g., rather than predominantly anti-autoimmune-disorder immunomodulatory effects). The anti-pathogen/anti-cancer immunomodulatory effects are associated with increases in at least some of Tₕ-1 cells, Tₕ-17 cells, and T_{reg} cells in the subject, and/or increases in at least some of IL-2, TNF-α, IFN-γ, IL1-β, IL-17, IL-22, and IL-9 in the subject. An increase in at least one of IL-17 and IL-22 can be greater than approximately 400% (e.g., about 500% or more).

In accordance with an aspect of the present disclosure, a phytochemical composition for facilitating or providing anti-autoimmune-disorder immunomodulatory effects in a subject includes an extract of mangosteen providing both alpha mangostin and gamma mangostin; an extract of defatted soybean; and an extract of defatted black sesame. The anti-autoimmune-disorder immunomodulatory effects are associated with decreases in at least some of Tₕ-1 cells, Tₕ-2 cells, Tₕ-17 cells, and T_{reg} cells; and/or decreases in at least some of IL-2, TNF-α, IFN-γ, IL-4, IL-6, IL-5, IL-17, IL-9, and IL-10. For instance, a reduction of TNF-α can be at least about 60%-70% (e.g., approximately 65%), or more than 80% (e.g., approximately 90%) depending upon embodiment details. A reduction of IL-17 can be greater than approximately 40%.

An anti-autoimmune-disorder composition can optionally include an extract of gotu kola, and wherein an amount of the extract of gotu kola is less than or equal to approximately 45% of the amount of a combination of the extract of mangosteen, the extract of defatted soybean, and the extract of defatted black sesame such that that the immunomodulatory effects provided by the composition are preferentially or predominantly anti-autoimmune-disorder immunomodulatory effects (e.g., rather than predominantly anti-pathogen immunomodulatory effects).

An anti-autoimmune-disorder composition can further include one of (a) a non-refined extract of gotu kola and a non-refined extract of guava to provide first anti-autoimmune-disorder immunomodulatory effects; and (b) a refined extract of gotu kola and a refined extract of guava to provide second anti-autoimmune-disorder immunomodulatory effects distinguishable from the first anti-autoimmune-disorder immunomodulatory effects (e.g., by way of measurement of cytokine levels within one or more subjects at one or more times, which indicate that the first and second anti-autoimmune-disorder compositions affect or modulate or reduce particular cytokine levels within the subject(s) in different, differentiable, or distinguishable manners). An amount of the non-refined extract of gotu kola or an amount of the refined extract of gotu kola can be less than or equal to 45% of the amount of a combination of the extract of mangosteen, the extract of defatted soybean, and the extract defatted black sesame such that that the immunomodulatory effects provided by the composition are preferentially/predominantly anti-autoimmune-disorder immunomodulatory effects.

In accordance with an aspect of the present disclosure, a process for selectively manufacturing one of a phytochemical anti-pathogen/anti-cancer composition or a phytochemical anti-autoimmune-disorder composition includes providing a predetermined amount of an extract of mangosteen; providing a predetermined amount of an extract of gotu kola; providing a predetermined amount of an extract of black sesame; and combining the extract of mangosteen with the extract of gotu kola and the extract of black sesame, wherein the predetermined of the extract of gotu kola is controlled relative to the predetermined amount of the extract of black sesame in order to selectively or preferentially produce either the anti-pathogen/anti-cancer composition or the anti-autoimmune-disorder composition.

In accordance with an aspect of the present disclosure, a process for selectively manufacturing a first phytochemical anti-autoimmune-disorder composition or a second phytochemical anti-autoimmune-disorder composition includes providing a predetermined amount of an extract of mangosteen; providing a predetermined amount of an extract of defatted black sesame; providing a predetermined amount of an extract of defatted soybean; and combining the extract of mangosteen, the extract of defatted black sesame, and the extract of defatted soybean with: (a) a predetermined amount of non-refined guava fruit juice powder and a predetermined amount of non-refined gotu kola fruit powder to produce the first phytochemical anti-autoimmune-disorder composition, or (b) a predetermined amount of refined guava fruit juice powder and a predetermined amount of refined gotu kola fruit powder to produce the second phytochemical anti-autoimmune-disorder composition, wherein the first anti-autoimmune-disorder composition supports or provides anti-autoimmune-disorder immunomodulatory effects that are distinguishable from anti-autoimmune-disorder immunomodulatory effects supported or provided by the second anti-autoimmune-disorder composition.

In accordance with an aspect of the present disclosure a process for supporting or providing fluctuating modulation of immune function to a subject, including anti-pathogen/anti-cancer immunomodulation and anti-autoimmune-disorder immunomodulation, includes administering an anti-pathogen/anti-cancer composition to the subject; and administering an anti-autoimmune-disorder compositions to the subject, wherein the administration of the anti-pathogen/anti-cancer composition is separated from the administration of the anti-autoimmune-disorder composition by at least about 2-6 hours.

In such an antipode fluctuating immunomodulation process, each of the anti-pathogen/anti-cancer composition and the anti-autoimmune-disorder compositions can be a phytochemical composition, wherein the anti-pathogen/anti-cancer composition includes an extract of mangosteen providing both alpha mangostin and gamma mangostin, an extract of defatted soybean, and an extract of gotu kola, and wherein the anti-autoimmune-disorder composition includes an extract of mangosteen providing both alpha mangostin and gamma mangostin, an extract of defatted soybean, and an extract of defatted black sesame. The anti-pathogen/anti-cancer composition can further include an extract of black sesame in an amount less than or equal to approximately 45% of an amount of a combination of the extract of mangosteen, the extract of defatted soybean, and the extract of gotu kola. The anti-autoimmune-disorder composition can further include an extract of gotu kola in an amount less than or equal to approximately 45% of an amount of a combination of the extract of mangosteen, the extract of defatted soybean, and the extract of defatted black sesame.

### BRIEF DESCRIPTION OF THE DRAWINGS

FIG. 1 is a representative graph illustrating an effect of a representative anti-pathogen composition in accordance with the present disclosure on the level of Tₕ-1, Tₕ-2, Tₕ-17, and T_{reg} cells in a group of test subjects relative to a placebo group.

FIG. 2 is a representative graph illustrating an effect of a representative first anti-autoimmune-disorder composition in accordance with the present disclosure on the level of Tₕ-1, Tₕ-2, Tₕ-17, and T_{reg} cells in a group of test subjects relative to a placebo group.

FIG. 3 is a representative graph illustrating an effect of a representative second anti-autoimmune-disorder composition in accordance with the present disclosure on the level of Tₕ-1, Tₕ-2, Tₕ-17, and T_{reg} cells in a group of test subjects relative to a placebo group.

### DETAILED DESCRIPTION

Unless explicitly stated otherwise, in the description herein the recitation of particular numerical values or value ranges is taken to be a recitation of particular approximate numerical values or approximate value ranges. For instance, a given numerical value or value range recited below should be interpreted or defined as an approximate numerical value or value range within +/−20%, +/−15%, +/−10%, or +/−1% to 10% (e.g., within +/−1%, 2%, 2.5%, 5%, or 7.5%) of the value or value range recited. The use of “/” herein is understood to mean “and/or” unless otherwise indicated.

Unless defined otherwise, all technical and scientific terms used herein have the same meanings as commonly understood by those having ordinary skill in the art of the present disclosure. As used herein, the term “set” corresponds to or is defined as a non-empty finite organization of elements that mathematically exhibits a cardinality of at least 1 (i.e., a set as defined herein can correspond to a singlet or single element set, or a multiple element set), in accordance with known mathematical definitions (for instance, in a manner corresponding to that described in An Introduction to Mathematical Reasoning: Numbers, Sets, and Functions, “Chapter 11: Properties of Finite Sets” (e.g., as indicated on p. 140), by Peter J. Eccles, Cambridge University Press (1998)). In general, an element of a set can include or be a composition, a compound, an ingredient, a process, a procedure, a physical parameter, a value, or an individual depending upon the type of set under consideration.

In the context of the present disclosure, the term “subject” refers to a human being or an animal (i.e., an organism belonging to the kingdom Animalia). The term phytochemical (or equivalently, nutraceutical or phytoceutical) as used herein refers to any substance, compound, or chemical that occurs naturally in plants (i.e., organisms belonging to the kingdom Plantae), including plants useable as food, or to any artificial, synthetic, or synthesizable substance, compound, or chemical having an identical, almost identical, or significantly similar chemical, physical, and/or structural properties to that which occurs naturally in plants, and which supports or provides identical, almost identical, or significantly similar biological effects in a living subject relative to its naturally occurring counterpart.

The terms “immune modulating,” “immunomodulating,” “immunomodulatory,” and “immunomodulation” as used herein refer to supporting or providing monitorable or measureable biological or physiological effects (e.g., changes in or maintenance of one or more biochemical markers) in a subject that facilitate, support, lead to, or induce beneficial changes in or maintenance of the state of the subject's health by way of aiding, adjusting, modifying, modulating, and/or varying the subject's immune system function at one or more times, such as by way of supporting the increase of or increasing the levels or expressions of one or more Tₕ cell populations and/or one or more cytokines, or supporting the decrease of or decreasing the levels or expressions of one or more Tₕ cell populations and/or cytokines within the subject.

The term “antipode immunomodulation” as used herein means that a first set of biological, or physiological effect(s) supported or provided by a first composition administered to a subject is absent from, or at least somewhat in opposition, generally opposite, or opposite to a second set of immunomodulatory effects supported or provided by a second composition administered to the subject. For instance, the first composition can support or provide immunomodulatory effects that result in an increase in particular cytokine levels, and the second composition can support or provide antipode immunomodulatory effects relative to the first composition, such as an absence of an increase in, or a lowering of, one or more of the cytokine levels that the first composition increases. Alternatively, the first composition can result in a decrease or preservation/maintenance of certain cytokine levels, whereas the second composition can result in an increase in one or more of such cytokine levels.

The terms “anti-pathogen” and “anti-pathogenic” refer to supporting, inducing, or providing at least one immunomodulatory effect that supports or results in the neutralization, destruction/eradication, or elimination of unhealthy or harmful viruses, bacteria, fungi, or foreign substances or matter within a subject's body. The term “anti-cancer” as used herein refers to supporting, inducing, or providing at last one immunomodulatory effect that acts against the spread, growth, or existence of cancer cells, for instance, an antiproliferative effect, apoptosis, or a cancer preventative effect.

The term “anti-autoimmune-disorder” refers to supporting, inducing, or providing at least one immunomodulatory effect that facilitates the reduction of, at least somewhat reduces, or reduces (a) an extent to which the subject's immune system chronically acts against or attacks their own bodily tissues and/or the their own physiological processes in a manner contrary or counter to the restoration or maintenance of good or optimal health, and/or (b) symptoms associated with an autoimmune or autoimmune-like disorder, dysfunction, or dysregulation. Representative examples of autoimmune or autoimmune-like disorders, dysfunction, or dysregulation that can be addressed by embodiments in accordance with the present disclosure include, but are not limited to, fibromyalgia, Crohn's disease, Inflammatory Bowel Disease (IBD), Irritable Bowel Syndrome (IBS), chronic fatigue syndrome (CFS), systemic lupus erythematosus (SLE), rheumatoid arthritis, psoriasis, diabetes, allergies (e.g., rhinitis, rash, or allergy based sinus congestion), oral cavity inflammation, asthma, hyperthyroidism, liver damage, gout, multiple sclerosis, and Parkinson's disease.

As further detailed below, various embodiments in accordance with the present disclosure are directed phytochemical compositions, including (a) compositions that can support or provide anti-pathogen/anti-cancer immonomodulatory effects, and (b) compositions that can support or provide anti-autoimmune-disorder immunomodulatory effects. While in some embodiments, an anti-pathogen/anti-cancer composition can be administered to a subject without consideration of an anti-autoimmune-disorder composition, or an anti-autoimmune disorder composition can be administered to the subject without consideration of an anti-pathogen/anti-cancer composition, in several embodiments a set of anti-pathogen/anti-cancer compositions and a set of anti-autoimmune disorder compositions are separately, serially, or sequentially administrable to the subject in a manner that supports or provides antipode immunomodulatory effects within the subject, in accordance with an antipode immunomodulation protocol.

Compositions in accordance with embodiments of the present disclosure include the following:
(a) a first or anti-pathogen/anti-cancer composition that can support or provide first or anti pathogen/anti-cancer immunomodulatory effects (e.g., both anti-pathogen and anti-cancer immunomodulatory effects), and which contains as its foundational, primary, or primarily active anti-pathogen components, constituents, or ingredients (i) a preparation of or an extract from Garcinia Mangostana L. (hereinafter referred to as “mangosteen”), including the xanthones alpha mangostin and gamma mangostin, (ii) a preparation of or an extract from defatted soybean or Glycine max, and (iii) a preparation of or an extract from Centella Asiatica L. (hereinafter referred to as “gotu kola”); and
(b) a second or anti-autoimmune-disorder composition that can support or provide second or anti-autoimmune-disorder immunomodulatory effects, and which contains as its foundational, primary, or primarily active anti-autoimmune-disorder components, constituents, or ingredients (i) a preparation of or an extract from mangosteen, including the xanthones alpha mangostin and gamma mangostin, (ii) a preparation of or an extract from defatted soybean or Glycine max, and (iii) a preparation of or an extract from defatted sesame or Sesamum indicum, which includes sesamin, sesamol, and/or sesamolin (hereinafter referred to as “sesame”).

For purpose of brevity and to aid understanding, an anti-pathogen/anti-cancer composition is simply referred to hereafter as an anti-pathogen composition.

Depending upon embodiment details, the anti-pathogen composition can include between 1%-100% by mass, weight, or volume of the aforementioned foundational, primary, or primarily active anti-pathogen composition components; and the anti-autoimmune disorder composition can include between 1%-100% by mass, weight, or volume of the aforementioned primary or primarily active anti-autoimmune-disorder composition components.

One or both of the anti-pathogen composition and the anti-autoimmune-disorder composition can also include additional or optional components, ingredients, or constituents, as further elaborated upon below. With respect to the primary components as well as any additional or optional components in each of the anti-pathogen composition and the anti-autoimmune-disorder composition, any given component can be prepared, extracted, washed, and/or purified in accordance with known techniques, such as alcohol extraction or supercritical extraction, in a manner readily understood by an individual having ordinary skill in the relevant art.

Each of the anti-pathogen composition and the anti-autoimmune-disorder composition can be in essentially any form that can be ingested or absorbed by or introduced into a subject. For example, the anti-pathogen composition and/or the anti-autoimmune-disorder composition can be in the form of a food product; a liquid or beverage product; one or more capsules, tablets, or pills; a powder; a suspension; an emulsion; a dietary supplement; an injection (parenteral); or another form. Furthermore, the anti-pathogen composition and the anti-autoimmune-disorder composition can both exist in the same form, or each can exist in a different form, depending upon embodiment details.

Representative Aspects of an Anti-Pathogen Composition

In various embodiments, the anti-pathogen composition includes as its primary or primarily active components, ingredients, or constituents a preparation of or an extract from mangosteen providing both alpha mangostin and gamma mangostin; a preparation of or an extract from defatted soybean; and a preparation of or an extract from gotu kola. In multiple embodiments, the extract from mangosteen includes or is mangosteen aril juice; the extract from defatted soybean includes or is soy protein islolate; and the extract from gotu kola includes or is centella asiatica juice powder. In several embodiments, the extract from mangosteen excludes constituents obtained from mangosteen exocarp or rind, because the rind includes tannins that can be hepatotoxic, and the rind can also carry pesticide residues.

The anti-pathogen composition can include one or more optional components or ingredients, such as an extract from guava; and/or an extract from sesame, which can be, but need not be, defatted, and which in some embodiments is at least partially defatted sesame. The optional extract from guava can include or be guava fruit juice powder; and the optional extract from sesame can include or be a black sesame extract (e.g., defatted black sesame extract).

In certain embodiments, additional optional anti-pathogen compound components or ingredients can include one or more other phytonutrients, phytochemicals, or nutraceuticals, for instance, a preparation or extract of one or more of turmeric or curcumin; cayenne; cinnamon; clove; nutmeg; garlic; ginger; green tea; pomegranate or raspberry (e.g., as a source of ellagic acid, a phenol antioxidant); blueberry leaf extract; honey; astragalus; one or more vitamins or minerals (for instance, one or more B vitamins (e.g., folic acid), selenium, magnesium, zinc, vitamin D, and/or vitamin C, such as liposomal vitamin C); methyl sulfonyl methane (MSM); one or more fatty acids (e.g., an omega-3 fatty acid, or coconut oil); olive leaf extract; an adaptogen (e.g., ginseng); a stilbenoid such as resveratrol or pterostilbene; active hexose correlated compound (AHCC); and/or another constituent or ingredient. In some embodiments, the anti-pathogen composition can be mixed or blended with a carrier substance (e.g., a liquid such as water or juice).

In a number of embodiments, the weight percentages or weight percentage ranges of the anti-pathogen composition's primary components relative to each other, in view of a total or 100% weight percentage of primary components exclusive of optional components, is as follows:
(a) extract from mangosteen: 10%-20%;
(b) extract from gotu kola: 50%-70%; and
(c) extract from defatted soybean: added or included in an amount relative to the combined weight percentage of the extract from mangosteen and the extract from gotu kola to provide 100% primary component weight percentage, depending upon the combined weight percentage of the extract from mangosteen and the extract from gotu kola.

With respect to the anti-pathogen composition's primary components, exclusive of optional components, the weight percentage of any individual primary component relative to the combined weight percentage of the other primary components can be selected, adjusted, or varied such that the sum or total of the individual primary component weight percentages equals 100%.

For instance, in a representative embodiment, the weight percentage of the extract of mangosteen relative to a combined weight percentage of the other primary components (i.e., the extract from gotu kola and the extract from defatted soybean) can be approximately 16.67%; the weight percentage of the extract from gotu kola relative to the combined weight percentage of the other primary components (i.e., the extract from mangosteen and the extract from defatted soybean) can be approximately 50%; and the weight percentage of the extract from defatted soybean relative to the combined weight percentage of the other primary components (i.e., the extract from mangosteen and the extract from gotu kola) can be approximately 33.33%.

In another representative embodiment, the weight percentage of the extract of mangosteen relative to the combined weight percentage of the extract from gotu kola and the extract from defatted soybean can be 10%; the weight percentage of the extract from gotu kola relative to the combined weight percentage of the extract from mangosteen and the extract from defatted soybean can be 70%; and the weight percentage of the extract from defatted soybean relative to the weight percentage of the extract from mangosteen and the extract from gotu kola can be 20%. Various other weight percentages of primary components relative to each of the other primary components are possible in accordance with the aforementioned primary component weight percentage ranges, in a manner readily understood by one having ordinary skill in the relevant art.

With respect to the primary components of the anti-pathogen composition, the extent or magnitude of the anti-pathogenic immunomodulatory effects provided by the combination of the extract from gotu kola and the extract from mangosteen are synergisticially enhanced compared to the immunomodulatory effects of the extract of goto kola considered in isolation or the effects of the extract of mangosteen considered in isolation. For instance, while centella asiatica by itself is known to give rise to immunostimulating effects (e.g., associated with pentacyclic triterpenes and saponins), and alpha mangostin and gamma mangostin are known to give rise to various anti-pathogenic/anti-cancer effects, the combination of an extract of gotu kola and an extract of mangosteen in accordance with embodiments of the present disclosure results in surprisingly and dramatically enhanced anti-pathogenic/anti-cancer immunomodulatory effects (e.g., associated with dramatic increases in IL-17 and/or IL-22 levels, as further elaborated upon below).

With respect to the total weight percentage of primary components in the anti-pathogen composition relative to the total weight percentage of additional/optional components, in various embodiments the primary components provide at least 30%-50% of the overall weight of the anti-pathogen composition, and typically provide at least or between 50%-80% of the overall weight of the anti-pathogen composition (depending upon the number and nature of additional/optional components, if any).

When an extract of sesame is included as an optional component in an anti-pathogen composition in accordance with an embodiment of the present disclosure, the weight percentage of the extract of sesame is controlled relative to the overall weight percentage of the anti-pathogen composition's primary components, and in particular can be controlled relative to the weight percentage of the extract of gotu kola, because an extract of sesame can support, promote, or provide anti-autoimmune-disease immunomodulation effects that act in an antipode manner relative to anti-pathogen immunomodulation effects, as further detailed below. More particularly, a relative weight percentage ratio between an extract of sesame and (a) a combination of an extract of mangosteen, an extract of gotu kola, and an extract of defatted soybean, or (b) an extract of gotu kola itself can influence, strongly influence, or determine whether a composition formulated or prepared in accordance with an embodiment of the present disclosure acts predominantly or mainly to provide anti-pathogen immunomodulation effects, or anti-autoimmune-disorder immunomodulation effects.

In general, an anti pathogen composition in accordance with an embodiment of the present disclosure that includes an extract of sesame as an optional component should exhibit, establish, or maintain a weight percentage of the anti-pathogen composition's primary components relative to a weight percentage of an extract of sesame of at least 55%-70% (e.g., approximately 60%) by weight of the primary components relative to the weight of the extract of sesame in order to predominantly or mainly support or provide anti-pathogen immunomodulatory effects. In other words, in an anti-pathogen composition in accordance with embodiments of the present disclosure, the weight of the extract of sesame, which is an optional component, should generally be less than or equal to 45% (e.g., approximately 40%) of the combined weight of the extract of mangosteen, the extract of gotu kola, and the extract of defatted soybean, such that the composition's immunomodulatory effects are predominantly anti-pathogenic in nature.

In specific embodiments, an anti-pathogen composition in accordance with an embodiment of the present disclosure that includes an extract of sesame as an optional component exhibits, establishes, or maintains a weight percentage of an extract of gotu kola relative to a weight percentage of an extract of sesame of at least 70% by weight in order to primarily or predominantly support or provide anti-pathogen immunomodulatory effects. That is, in specific embodiments of an anti-pathogen composition in accordance with the present disclosure, the weight of the extract of sesame, which is an optional component, should be less than or equal to 30% of the weight of the extract of gotu kola, such that the composition's immunomodulatory effects are primarily or predominantly anti-pathogenic in nature.

In one representative embodiment, the overall weight percentage of the anti-pathogen composition (exclusive of any weight associated with a capsule or carrier medium) includes 50% by weight of the primary anti-pathogen composition components (i.e., an extract from mangosteen, an extract from gotu kola, and an extract from defatted soybean), and also 50% by weight of a combination of an extract from guava and an extract from sesame.

In such an embodiment, the weight percentages of the primary and optional components relative to each other can be established or adjusted as follows, in view of the aforementioned weight percentage relationship between an extract of gotu kola and an extract of sesame:
(a) extract from mangosteen: 5%-10%;
(b) extract from gotu kola: 25%-35%;
(d) extract from defatted soybean: added or included in an amount relative to the combined weight percentage of the extract from mangosteen and the extract from gotu kola to sum to 50% of the total weight percentage of the anti-pathogen composition (e.g., between 5%-20%);
(e) extract from sesame: up to 30% of the weight of the extract from gotu kola; and
(f) extract from guava: added or included in an amount relative to the combined weight percentage of the extract from mangosteen, the extract from gotu kola, the extract from defatted soybean, and the extract from sesame to sum to 100% total weight percentage of the anti-pathogen composition.

In another representative embodiment, the overall weight of the anti-pathogen composition (exclusive of any weight associated with a capsule or carrier medium) includes 75% by weight of the primary anti-pathogen composition components (i.e., an extract from mangosteen, an extract from gotu kola, and an extract from defatted soybean), and also 25% by weight of a combination of an extract from guava and an extract from sesame. Thus, in this embodiment, the weight percentages of the primary and optional components relative to each other can be established or adjusted as follows, in view of the aforementioned weight percentage relationship between an extract of gotu kola and an extract of sesame:
(a) extract from mangosteen: 7.5%-15%;
(b) extract from gotu kola: 37.5%-52.5%;
(c) extract from defatted soybean: added or included in an amount relative to the combined weight percentage of the extract from mangosteen and the extract from gotu kola to sum to 75% of the total weight percentage of the anti-pathogen composition, (e.g., between 7.5%-30%);
(d) extract from sesame: up to 30% of the weight of the extract from gotu kola; and
(e) extract from guava: added or included in an amount relative to the combined weight percentage of the extract from mangosteen, the extract from gotu kola, the extract from defatted soybean, and the extract from sesame to sum to 100% total weight percentage of the anti-pathogen composition.

The anti-pathogen composition can support, facilitate, or provide anti-pathogen immunomodulation effects by way of inducing an alteration or change in the quantity of white blood cells, and/or proteins or signaling molecules secreted by white blood cells within biological tissue of a subject. More specifically, an anti pathogen composition according to an embodiment of the present disclosure can support, lead to, or provide an alteration or change in the number of white blood cells, such as, for example, phagocytes, Natural Killer cells (NK cells), cytotoxic T-cell, B cell, plasma cells, Tₕ cells, Tₕ-1 cells, Tₕ-2 cells, Tₕ-17 cells, and T_{reg} cells in a subject to whom the anti-pathogen composition is administered (e.g., by way of oral ingestion). Additionally, the anti-pathogen composition according to the present disclosure can support, facilitate, lead to, or provide an alteration or change in an amount or quantity of signaling molecules (cytokines) secreted by white blood cells including, but not limited to, tumor necrosis factor (TFN-α), interferon (IFN-γ), and interleukin (IL) 2, 5, 6, 9, 10, 13, 17, and 22.

In some embodiments, the anti-pathogen composition can support, facilitate, lead to, or provide an increased expression of Tₕ-1 as determined by the expression of cytokines of IL-2, TNF-α, and IFN-γ. For instance, in an embodiment, the anti-pathogen composition can support, facilitate, lead to, or provide an increased, changed, or altered expression of IL-2, TNF-α, and IFN-γ in a subject to whom the anti-pathogen composition is administered as compared to the level of IL-2, TNF-α, and IFN-γ in a control subject to whom the anti-pathogen composition is not administered (e.g., a control subject to whom a placebo is administered) by about 20%-40% for IL-2; about 50%-80% for TNF-α; and about 60%-90% for IFN-γ.

In some embodiments, the anti-pathogen composition can support, facilitate, lead to, or provide an alteration or change of expression of Tₕ-2 as determined by expression of cytokines IL-1β, IL-6, IL-13, and IL-5. In an embodiment, the anti-pathogen composition can increase expression of IL-1β by about 5%-20%; change (e.g., decrease) expression of IL-6 and/or IL-13 by up to about 10%; and decrease expression of IL-5 by about 40%-60% in a subject to whom the anti-pathogen composition is administered compared to a control subject to whom the anti-pathogen composition is not administered.

In various embodiments, the anti-pathogen composition can support, facilitate, lead to, or provide a dramatic alteration or change of expression of Tₕ-17 as determined by IL-17 and/or IL-22 levels. In an embodiment, the anti-pathogen composition can support, facilitate, lead to, or provide an increase of expression of IL-17 levels by about 450%-500%, and an increase of expression of IL-22 levels by about 525%-575% in a subject to whom the anti-pathogen composition is administered compared to a control subject to whom the anti-pathogen composition is not administered. Thus, an anti-pathogen composition in accordance with an embodiment of the present disclosure can support or provide an increase in each of IL-17 and IL-22 levels by at least about 400%, or by about 500%.

In several embodiments, the anti-pathogen composition can further support, facilitate, lead to, or provide an alteration or change of expression of T_{reg} as determined by the levels of cytokines IL-9 and/or IL-10. In an embodiment, the anti-pathogen composition can increase IL-9 levels by about 100%-115%; and can increase IL-10 levels by up to about 5-10% in a subject to whom the anti-pathogen composition is administered compared to a control subject to whom the anti-pathogen composition is not administered.

A single dose of the anti-pathogen composition useful to support, facilitate, lead to, or provide anti-pathogenic immunomodulation by way of inducing a change in the quantity of white blood cells, proteins and signaling molecules secreted by white blood cells within biological tissue of a subject (e.g., of an average size child or adult human being, or a reduced mass child or adult human being suffering from a health condition or immune dysfunction, although other living organisms can be treated) can provide at least approximately 25-50 mg of the anti-pathogen composition's primary or primarily active components, and in various situations, up to 1000 mg of the anti-pathogen composition's primary or primarily active components. In some embodiments, the anti-pathogen composition can include about 50 mg to about 500 mg of a combination of the extract of mangosteen, the extract of gotu kola, and the extract of defatted soybean per dosage unit (capsule, pill, syrup, etc . . . ). In some embodiments, a daily dose of an anti-pathogen composition formulated in accordance with the present disclosure provides at least approximately 50 mg to 2,000 mg of a combination of an extract of mangosteen, an extract of gotu kola, and an extract of defatted soybean. Any daily dose can be divided into sub-doses, for administration in accordance with an administration protocol disclosed herein, for instance, based upon time of day, meal consumption, and/or health condition. For example, a daily dose of the anti-pathogen composition can be divided and administered in the form of capsules by consuming, for example, 2 capsules twice per day, wherein each capsule contains 25% of a total daily dosage, and each capsule includes a combination of an extract of mangosteen, an extract of gotu kola, and an extract of defatted soybean for supporting, facilitating, or providing anti-pathogen immunomodulation in accordance with an embodiment of the present disclosure. It will, however, be understood by a person of ordinary skill in the art that alternative dosages (e.g., 1, 3, 4, 5, 6, or more capsules per day) can be suggested or administered.

Representative Aspects of an Anti-Autoimmune-Disorder Composition

In various embodiments, the anti-autoimmune-disorder composition includes as its primary or primarily active components, ingredients, or constituents a preparation of or an extract from mangosteen providing both alpha mangostin and gamma mangostin; a preparation of or an extract from defatted soybean, and a preparation of or an extract from defatted sesame or at least partially defatted sesame. In multiple embodiments, the extract from mangosteen includes or is mangosteen aril juice; the extract from defatted soybean includes or is soy protein isolate; and the extract from defatted sesame includes or is defatted black sesame extract, or at least partially defatted black sesame extract. In several embodiments, the extract from mangosteen excludes constituents obtained from mangosteen exocatp or rind, for reasons set forth above.

In some embodiments, the anti-autoimmune-disorder composition can additionally or optionally include an extract from guava, and an extract from gotu kola. The additional or optional extract from guava can include or be guava fruit juice powder, which in a number of embodiments can be refined guava fruit juice powder (e.g., refined by way of a washing procedure, for instance, involving hexane washing following alcohol extraction); and the additional or optional extract from gotu kola can include or be gotu kola fruit powder, which in a number of embodiments can be refined gotu kola fruit powder (e.g., refined by way of a washing procedure, for instance, involving hexane washing following alcohol extraction).

In embodiments that additionally include an extract of guava in the form of refined guava juice powder produced by way of alcohol extraction followed by hexane washing and an extract of gotu kola in the form of refined gotu kola fruit powder provided by way of alcohol extraction followed by hexane washing, it was serendipitously discovered that the immunomodulatory effects of the anti-autoimmune-disorder composition differed or were distinguishable from the immunomodulatory effects provided by embodiments that lacked refined guava juice powder and refined gotu kola fruit powder. Such differences in immunomodulatory effects provided by anti-autoimmune-disorder compositions that include refined guava juice powder and refined gotu kola fruit powder versus anti-autoimmune-disorder compositions that exclude refined guava juice powder and refined gotu kola fruit powder (or which include unrefined guava juice powder and unrefined gotu kola fruit powder) enable the anti-autoimmune-disorder composition's immunomodulatory effects to be differentiated, customized, or tailored to a certain extent with regard to addressing different categorical types of autoimmune dysfunction or dysregulation, as further described in detail below.

In certain embodiments, an anti-autoimmune disorder composition can further include a set of additional or other optional anti-autoimmune-disorder components or ingredients, such as one or more other phytonutrients, phytochemicals, or nutraceuticals, for instance, a preparation or extract of one or more of turmeric or curcumin; cayenne; cinnamon; green tea; artichoke or artichoke leaf; blueberry leaf extract; one or more vitamins and/or minerals (e.g., vitamin C and/or vitamin E); methyl sulfonyl methane (MSM); one or more fatty acids (e.g., an omega-3 fatty acid, or coconut oil); one or more enzymes such as serrapeptase; grape seed extract; a stilbenoid such as resveratrol or pterostilbene; an adptogen; a probiotic; and/or another constituent or ingredient. In specific embodiments, one or more types of optional ingredients can be selected for inclusion in an anti-autoimmune-disorder composition based upon subject profile and/or a type of autoimmune disorder, dysregulation, or dysfunction under consideration. In some embodiments, the anti-autoimmune-disorder composition can be mixed or blended with a carrier substance (e.g., a liquid such as water or juice).

In a number of embodiments, the weight percentages or weight percentage ranges of the anti-autoimmune-disorder composition's primary components relative to each other, in view of a total or 100% weight percentage of primary components exclusive of other or optional components, is as follows:
(a) extract from mangosteen: 10%-20%;
(b) extract from defatted sesame: 50%-70%; and
(c) extract from defatted soybean: added or included in an amount relative to the combined weight percentage of the extract from mangosteen and the extract from sesame to provide 100% primary component weight percentage, depending upon the combined weight percentage of the extract from mangosteen and the extract from defatted sesame.

With respect to the anti-auto immune-disorder composition's primary components, exclusive of optional components, the weight percentage of any individual primary component relative to the combined weight percentage of the other primary components can be selected, adjusted, or varied such that the sum or total of the individual primary component weight percentages equals 100%.

For instance, in a representative embodiment, the weight percentage of the extract of mangosteen relative to a combined weight percentage of the other primary components (i.e., the extract from defatted sesame and the extract from defatted soybean) can be approximately 16.67%; the weight percentage of the extract from defatted sesame relative to the combined weight percentage of the other primary components (i.e., the extract from mangosteen and the extract from defatted soybean) can be approximately 50%; and the weight percentage of the extract from defatted soybean relative to the combined weight percentage of the other primary components (i.e., the extract from mangosteen and the extract from defatted sesame) can be approximately 33.33%.

In another representative embodiment, the weight percentage of the extract of mangosteen relative to the combined weight percentage of the extract from defatted sesame and the extract from defatted soybean can be 10%; the weight percentage of the extract from defatted sesame relative to the combined weight percentage of the extract from mangosteen and the extract from defatted soybean can be 70%; and the weight percentage of the extract from defatted soybean relative to the weight percentage of the extract from mangosteen and the extract from defatted sesame can be 20%. Various other weight percentages of primary components relative to each of the other primary components of the anti-autoimmune-disorder composition are possible in accordance with the aforementioned primary component weight percentage ranges, in a manner readily understood by one having ordinary skill in the relevant art.

With respect to the primary components of the anti-autoimmune-disorder composition, the extent or magnitude of the anti-autoimmune-disorder immunomodulatory effects provided by the combination of the extract from defatted sesame and the extract from mangosteen are synergistically enhanced compared to the immunomodulatory effects of the extract of defatted sesame considered in isolation or the effects of the extract of mangosteen considered in isolation. For instance, sesamin by itself is known to inhibit Tocopherol-ω-hydroxylation, which can augment γ-tocopherol and γ-tocotrienol metabolism by preventing their degradation to thereby increase the levels of these vitamers of vitamin E. Moreover, vitamin E is known to be a cellular and lysosomal membrane stabilizer, which can play a role in mediating or mitigating the effects or progression of autoimmune processes. Mangosteen constituents by themselves are known to facilitate modulation or inhibition of inflammatory processes. For instance, gamma mangostin is known to inhibit the activities of cyclooxygenase-1 (COX-1) and inducible cyclooxygenase-2 (COX-2), which play a role in inflammatory processes. The combination of an extract of defatted sesame and an extract of mangosteen in accordance with embodiments of the present disclosure synergistically results in surprising and significantly enhanced anti-autoimmune-disorder immunomodulatory effects compared to their activities in isolation (e.g., associated with dramatically decreased levels of TNF-α, and/or very significantly decreased levels of one or more of IFN-γ, IL-4, IL-5, and IL-17, as further detailed below).

With respect to the total weight percentage of primary components in the anti-autoimmune-disorder composition relative to the total weight percentage of additional/optional components, in various embodiments the primary components provide at least 30-50% of the overall weight of the anti-autoimmune-disorder composition, and typically provide at least or between 50%-80% of the overall weight of the anti-autoimmune-disorder composition (depending upon the number and nature of additional/optional components, if any).

When an extract of gotu kola is included as an optional component in an anti-autoimmune-disorder composition, the weight percentage of the extract of gotu kola is controlled relative to the overall weight percentage of the anti-autoimmune-disorder composition's primary components, and in particular can be controlled relative to the weight percentage of the extract of defatted sesame, because an extract of gotu kola can support, promote, or provide anti-pathogen immunomodulation effects that act in an antipode manner relative to anti-autoimmune-disorder immunomodulation effects, as further detailed below. More particularly, a relative weight percentage ratio between an extract of gotu kola and (a) a combination of an extract of mangosteen, an extract of defatted sesame, and an extract of defatted soybean, or (b) an extract of defatted sesame by itself can influence, strongly influence, or determine whether a composition formulated or prepared in accordance with an embodiment of the present disclosure acts predominantly or mainly to provide anti-autoimmune-disorder immunomodulation effects, or anti-pathogen immunomodulation effects.

In general, an anti-autoimmune-disorder composition in accordance with an embodiment of the present disclosure that includes an extract of gotu kola as an optional component should exhibit, establish, or maintain a weight percentage of the anti-autoimmune-disorder composition's primary components relative to a weight percentage of an extract of gotu kola of at least 55%-70% (e.g., approximately 60%) by weight of the primary components relative to the weight of the extract of gotu kola in order to predominantly or mainly support or provide anti-autoimmune-disorder immunomodulatory effects. In other words, in an anti-autoimmune-disorder composition in accordance with embodiments of the present disclosure, the weight of the extract of gotu kola, which is an optional component, should generally be less than or equal to 45% (e.g., approximately 40%) of the combined weight of the extract of mangosteen, the extract of defatted sesame, and the extract of defatted soybean, such that the composition's immunomodulatory effects are predominantly anti-autoimmune-disorder in nature.

In specific embodiments, an anti-autoimmune-disorder composition in accordance with an embodiment of the present disclosure that includes an extract of gotu kola as an optional component exhibits, establishes, or maintains a weight percentage of an extract of defatted sesame relative to a weight percentage of an extract of gotu kola of at least 70% by weight in order to primarily or predominantly support or provide anti-autoimmune-disorder immunomodulatory effects. That is, in specific embodiments of an anti-autoimmune-disorder composition in accordance with the present disclosure, the weight of the extract of gotu kola, which is an optional component, should be less than or equal to 30% of the weight of the extract of defatted sesame, such that the composition's immunomodulatory effects are primarily or predominantly anti-autoimmune-disorder in nature.

In one representative embodiment, the overall weight percentage of the anti-autoimmune-disorder composition (exclusive of any weight associated with a capsule or carrier medium) includes 50% by weight of the primary anti-autoimmune-disorder composition components (i.e., an extract from mangosteen, an extract from defatted sesame, and an extract from defatted soybean), and also 50% by weight of a combination of an extract from guava and an extract from gotu kola.

In such an embodiment, the weight percentages of the primary and optional components relative to each other can be established or adjusted as follows, in view of the aforementioned weight percentage relationship between an extract of defatted sesame and an extract of gotu kola:
(a) extract from mangosteen: 5%-10%;
(b) extract from defatted sesmae: 25%-35%;
(c) extract from defatted soybean: added or included in an amount relative to the combined weight percentage of the extract from mangosteen and the extract from gotu kola to sum to 50% of the total weight percentage of the anti-autoimmune-disorder composition (e.g., between 5%-20%);
(d) extract from gotu kola: up to 30% of the weight of the extract from defatted sesame; and
(e) extract from guava: added or included in an amount relative to the combined weight percentage of the extract from mangosteen, the extract from defatted sesame, the extract from defatted soybean, and the extract from gotu kola to sum to 100% total weight percentage of the anti-autoimmune-disorder composition.

In another representative embodiment, the overall weight of the anti-pathogen composition (exclusive of any weight associated with a capsule or carrier medium) includes 75% by weight of the primary anti-autoimmune-disorder composition components (i.e., an extract from mangosteen, an extract from defatted sesame, and an extract from defatted soybean), and also 25% by weight of a combination of an extract from guava and an extract from gotu kola. Thus, in this embodiment, the weight percentages of the primary and optional components relative to each other can be established or adjusted as follows, in view of the aforementioned weight percentage relationship between an extract of defatted sesame and an extract of gotu kola:
(a) extract from mangosteen: 7.5%-15%;
(b) extract from defatted sesame: 37.5%-52.5%;
(c) extract from defatted soybean: added or included in an amount relative to the combined weight percentage of the extract from mangosteen and the extract from defatted sesame to sum to 75% of the total weight percentage of the anti-autoimmune-disorder composition, (e.g., between 7.5%-30%);
(d) extract from gotu kola: up to 30% of the weight of the extract from defatted sesame; and
(e) extract from guava: added or included in an amount relative to the combined weight percentage of the extract from mangosteen, the extract from gotu kola, the extract from defatted soybean, and the extract from sesame to sum to 100% total weight percentage of the anti-autoimmune-disorder composition.

An anti-autoimmune-disorder composition can support, facilitate, or provide anti-autoimmune-disorder immunomodulation effects by way of inducing an alteration or change in the quantity of white blood cells; and/or proteins or signaling molecules secreted by white blood cells within biological tissue of a subject. More specifically, an anti-autoimmune-disorder composition according to an embodiment of the present disclosure can support, lead to, or provide an alteration or change in the number of white blood cells, such as, for example, Tₕ-1 cells, Tₕ-2 cells, Tₕ-17 cells, and T_{reg} cells in a subject to whom the anti-autoimmune-disorder composition is administered (e.g., by way of oral ingestion). Additionally, the anti-autoimmune-disorder composition according to the present disclosure can support, facilitate, lead to, or provide an alteration or change in an amount or quantity of signaling molecules (cytokines) secreted by white blood cells including, but not limited to, at least some of tumor necrosis factor (TFN-α), interferon (IFN-γ), and interleukin (IL) 1β, 2, 6, 9, 10, 13, 17, and 22.

In some embodiments, the anti-autoimmune-disorder composition can support, facilitate, lead to, or provide a decreased expression of Tₕ-1 as determined by the expression of cytokines of IL-2, TNF-α, and IFN-γ. For instance, in an embodiment, the anti-autoimmune-disorder composition can support, facilitate, lead to, or provide a decreased expression of IL-2, TNF-α, and IFN-γ in a subject to whom the anti-autoimmune-disorder composition is administered as compared to the level of IL-2, TNF-α, and IFN-γ in a control subject to whom the anti-autoimmune-disorder composition is not administered (e.g., a control subject to whom a placebo is administered) by about 35%-45% for IL-2; about 80%-95% for TNF-α; and about 5% for IFN-γ.

In some embodiments, the anti-autoimmune-disorder composition can support, facilitate, lead to, or provide an alteration or change of expression of Tₕ-2 as determined by expression of one or more of cytokines IL-1β, IL-4, IL-5, IL-6, and IL-13. In an embodiment, the anti-autoimmune-disorder composition can decrease expression of IL-1β by about 5%; decrease expression of IL-6 and/or IL-13 by about 20%-30%; and decrease expression of IL-13 by up to about 5% in a subject to whom the anti-autoimmune-disorder composition is administered compared to a control subject to whom the anti-anti-autoimmune-disorder composition is not administered. In another embodiment, the anti-autoimmune-disorder composition can decrease expression of IL-4 by about 35%, and decrease expression of IL-5 by about 50% in a subject to whom the anti-autoimmune-disorder composition is administered compared to a control subject to whom the anti-autoimmune-disorder composition is not administered.

In various embodiments, the anti-autoimmune-disorder composition can support, facilitate, lead to, or provide an alteration or change of expression of Tₕ-17 as determined by IL-17 level. In an embodiment, the anti-autoimmune-disorder composition can support, facilitate, lead to, or provide a decrease of expression of IL-17 levels by about 40%-50% in a subject to whom the anti-autoimmune-disorder composition is administered compared to a control subject to whom the anti-autoimmune-disorder composition is not administered.

In several embodiments, the anti-autoimmune-disorder composition can further support, facilitate, lead to, or provide an alteration or change of expression of T_{reg} as determined by the levels of cytokines IL-9 and/or IL-10. In an embodiment, the anti-autoimmune-disorder composition can decrease IL-9 and/or IL-10 levels by about 10%-20% in a subject to whom the anti-autoimmune-disorder composition is administered compared to a control subject to whom the anti-autoimmune-disorder composition is not administered.

The dramatic reduction of TNF-α and the significant or very significant reduction of IL-6 and IL-17 provided by an anti-autoimmune-disorder composition in accordance with an embodiment of the present disclosure facilitates, is associated with, or provides a reduction in the severity of symptoms associated with autoimmune disorders, dysregulation, or dysfunction.

A single dose of the anti-autoimmune-disorder composition useful to support, facilitate, lead to, or provide anti-autoimmune-disorder immunomodulation by way of inducing a change in the quantity of white blood cells, proteins and signaling molecules secreted by white blood cells within biological tissue of a subject (e.g., of an average size child or adult human being, or a reduced mass child or adult human being suffering from a health condition or immune dysfunction, although other living organisms can be treated) can provide at least approximately 25-50 mg of the anti-autoimmune-disorder composition's primary or primarily active components, and in various situations, up to 500 mg of the anti-autoimmune-disorder composition's primary or primarily active components. In some embodiments, the anti-autoimmune-disorder composition can include about 50 mg to about 400 mg of a combination of the extract of mangosteen, the extract of defatted sesame, the extract of defatted soybean, and possibly the extract of gotu kola (e.g., refined or non-refined extract of gotu kola) and the extract of guava (e.g., refined or non-refined extract of guava) per dosage unit (capsule, pill, syrup, etc.). In some embodiments, a daily dose of an anti-autoimmune-disorder composition formulated in accordance with the present disclosure provides at least approximately 50 mg to 2,000 mg of a combination of an extract of mangosteen, an extract of defatted sesame, an extract of defatted soybean, and possibly an extract of gotu kola (e.g., refined or non-refined extract of gotu kola) and an extract of guava (e.g., refined or non-refined extract of guava). Any daily dose can be divided into sub-doses, for administration in accordance with an administration protocol disclosed herein, for instance, based upon time of day, meal consumption, and/or health condition. For example, a daily dose of the anti-autoimmune-disorder composition can be divided and administered in the form of capsules by consuming, for example, 2 capsules twice per day, wherein each capsule contains 25% of a total daily capsule dosage, and each capsule includes a combination of an extract of mangosteen, an extract of defatted sesame, an extract of defatted soybean, and possibly an extract of gotu kola (e.g., refined or non-refined extract of gotu kola) and an extract of guava (e.g., refined or non-refined extract of guava) for supporting, facilitating, or providing anti-autoimmune-disorder immunomodulation in accordance with an embodiment of the present disclosure. It will, however, be understood by a person of ordinary skill in the art that alternative dosages (e.g., 1, 3, 4, 5, 6, or more capsules per day) can be suggested or administered.

### EXAMPLES

### Example 1

An anti-pathogen composition in the form of a capsule was provided in accordance with the present disclosure. Each capsule included 10% by weight mangosteen extract, 20% by weight of gotu kola extract, 20% by weight of defatted soybean extract, 30% by weight of black sesame extract, and 20% by weight of guava extract. The capsules were formulated or manufactured by standard methods, processes, and/or techniques known to a person of ordinary skill in the art.

The anti-pathogen composition capsules were administered to six human volunteers forming a group of test subjects in a dosage of 4 capsules per day for 15 days, in accordance with an administration protocol of 2 capsules in the morning before breakfast, and 2 capsules in the evening before bedtime. Six other human volunteers forming a placebo group were provided a dosage of 4 placebo capsules (containing corn starch) per day for 15 days, in accordance with the same administration protocol (i.e., 2 placebo capsules in the morning before breakfast, and 2 placebo capsules in the evening before bedtime).

On day 15, blood cytokine levels of individuals in the test subject group and the placebo group were tested using flow cytometry (FlowCytomix™). Levels of IL-2, TNF-α, and IFN-γ indicative of the amount of Tₕ-1 cells; IL-1β, IL-6, IL-13, and IL-5 indicative of the level of Tₕ-2 cells; and IL-17/IL-22 and IL-9/IL-10 indicative of the amount of Tₕ-17 and T_{reg} cells, respectively, were tested.

Results

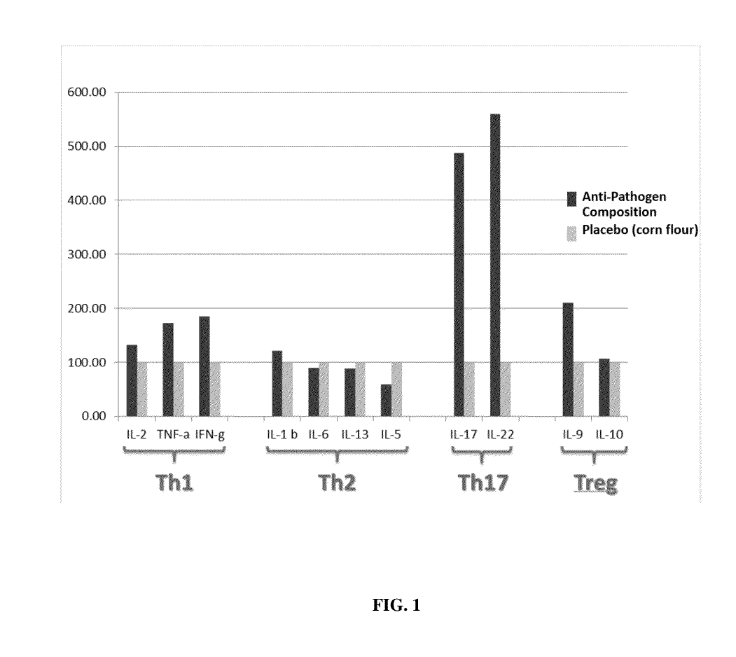

FIG. 1 shows an effect of this anti-pathogen composition according to the present disclosure (comprising an extract from mangosteen, an extract from gotu kola, an extract from defatted soybean, an extract from black sesame, and an extract from guava) versus a placebo on the level of Tₕ-1, Tₕ-2, Tₕ-17, and T_{reg} cells, indicative of anti-pathogen immune modulation. Placebo group results were normalized to 100.

Upon administration of the anti-pathogen composition, the amount of Tₕ-1 cells significantly or noticeably increased in the test subject group, as compared to the placebo group. The amount of Tₕ-2 cells in the test subject group decreased as compared to the placebo group. Furthermore, as illustrated in FIG. 1, the quantity of Tₕ-17 cells and Tₕ-22 cells in the test subject group, who took the anti-pathogen composition according to the present disclosure, dramatically increased by approximately fivefold as compared to the placebo group.

The anti-pathogen immune modulating composition provided by the present disclosure surprisingly appears to increase Tₕ-17 cells approximately fivefold. Thus, the results obtained in Example 1 suggest that the anti-pathogen immune modulating composition according to the present disclosure can support, facilitate, or provide anti-pathogen as well as cancer cell reduction. The level of T_{reg} cells in the test subject group is significantly higher than in the placebo group, particularly as evidenced by an approximate doubling in the level of IL-9 in subjects to whom the anti-pathogen composition was administered as compared to the placebo group.

Conclusion

Experiments in example 1 indicate that an anti-pathogen composition provided by the present disclosure can support, facilitate, or provide anti-pathogen and anti-cancer immunomodulation specifically by affecting Tₕ-1, Tₕ-2, Tₕ-17, and T_{reg} cells. In addition, the immune modulating composition according to the present disclosure can support, facilitate, or provide both pathogen reduction and cancer cell reduction through a significant increase in or modulation of Tₕ-1 cells, and a very strong regulation or modulation of Tₕ-17 and Tₕ-22 cells as evidenced by an approximate five-fold increase of Tₕ-17 and Tₕ-22 cells, which are capable of fighting pathogens, foreign matter, and cancer cells beyond Tₕ-1 and Tₕ-2 cell capability. In other words, an anti-pathogen composition provided in accordance with the present disclosure can support, improve, enhance, or modulate immune system function to fight pathogens, foreign matter, and/or cancer cells.

### Example 2

A first anti-autoimmune-disorder composition in the form of a capsule was provided to a total of 6 subjects in accordance with the present disclosure. The first anti-autoimmune-disorder capsule included 10% by weight of an extract of mangosteen, 20% by weight of an extract of defatted soybean in the form of isolated soy protein, 20% by weight of an extract of defatted black sesame, 30% by weight of an extract of gotu kola in the form of non-refined gotu kola fruit powder, and 20% by weight of an extract of guava in the form of non-refined guava fruit juice powder. The first anti-autoimmune-disorder capsules were formulated or manufactured by standard methods, processes, and/or techniques known to a person of ordinary skill in the art.

The first anti-autoimmune-disorder composition capsules of Example 2 were administered to six human volunteers forming a test subject group in a dosage of 4 capsules per day for 15 days, in accordance with an administration protocol of 2 capsules in the morning before breakfast, and 2 capsules in the evening before bedtime. Six other human volunteers forming a placebo group were provided a dosage of 4 placebo capsules (containing corn starch) per day for 15 days, in accordance with the same administration protocol (i.e., 2 placebo capsules in the morning before breakfast, and 2 placebo capsules in the evening before bedtime).

On day 15, blood cytokine levels of the individuals within the test subject group and the placebo group were tested using flow cytometry (FlowCytomix™). Levels of IL-2, TNF-α, and IFN-γ indicative of the amount of Tₕ-1 cells; IL-1β, IL-6, and IL-13 indicative of the level of Tₕ-2 cells; and IL-17/IL-22 and IL-9/IL-10 indicative of the amount of Tₕ-17 and T_{reg} cells, respectively, were tested.

Results

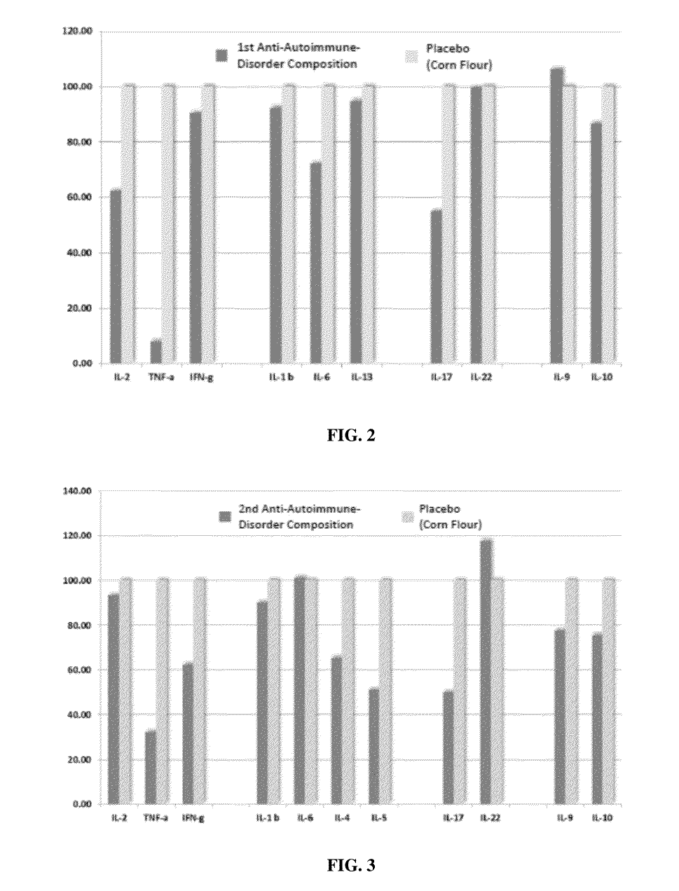

FIG. 2 shows an effect of the first anti-autoimmune-disorder composition according to present disclosure on the level of Tₕ-1, Tₕ-2, Tₕ-17, and T_{reg} cells. Placebo group results were normalized to 100. As a result of the administration of the first anti-autoimmune-disorder composition, the level of Tₕ-1 cells in the test subject group significantly decreased as compared to the placebo group. In particular, the level of TNF-α dramatically decreased (by more than 80%, e.g., approximately 90%). The level of Tₕ-2 cells in the test subject group slightly or moderately decreased as compared to the placebo group; IL-6 in particular decreased significantly (by more than 20%, e.g., approximately 25%-30%) in the test subject group compared to the placebo group. As further illustrated by FIG. 2, the level of Tₕ-17 significantly decreased in test subject group volunteers taking the anti-autoimmune-disorder composition compared to the placebo group, particularly as evidenced by a very significant drop in IL-17 level. The level of T_{reg} cells in the test subject group was approximately the same, or slightly decreased, in the test subject group compared to the placebo group.

Conclusion

Experiments in example 2 indicated that the first anti-autoimmune-disorder composition can support or facilitate the modulation of immune cell levels, specifically by significantly decreasing the levels of Tₕ-1 and Tₕ-17 cells, and at least slightly or moderately deceasing the level of Tₕ-2 cells. Thus, the first anti-autoimmune-disorder composition can support or provide significantly or very significantly reduced levels of pro-inflammatory cytokines that are known to play a role in inflammatory, auto-immune, or autoimmune-like conditions. These results support the first anti-autoimmune-disorder composition's ability to facilitate or provide anti-autoimmune-disorder immunomodulation effects, particularly with respect to dramatically decreasing levels of TNF-α, which plays a key role in regulating the inflammatory response process in autoimmune disorders such as rheumatoid arthritis; and significantly decreasing IL-6 and IL-17 levels, which is known to act in a pro-inflammatory manner in asthma. Consequently, the first anti-autoimmune-disorder composition appears well suited for supporting, facilitating, or providing at least anti-rheumatoid-arthritis and/or asthma immunomodulation.

### Example 3

A second anti-autoimmune-disorder composition in the form of a capsule was provided to a total of 6 subjects in accordance with the present disclosure. The first anti-autoimmune-disorder capsule included 10% by weight of an extract of mangosteen, 20% by weight of an extract of defatted soybean in the form of isolated soy protein, 20% by weight of an extract of defatted black sesame, 30% by weight of an extract of gotu kola in the form of refined gotu kola fruit powder (i.e., as an alcohol extract that had been hexane washed), and 20% by weight of an extract of guava in the form of refined guava fruit juice powder (i.e., as an alcohol extract that had been hexane washed). The second anti-autoimmune-disorder capsules were formulated or manufactured by standard methods, processes, and/or techniques known to a person of ordinary skill in the art.

The second anti-autoimmune-disorder composition capsules of Example 3 were administered to six human volunteers forming a test subject group in a dosage of 4 capsules per day for 15 days, in accordance with an administration protocol of 2 capsules in the morning before breakfast, and 2 capsules in the evening before bedtime. Six other human volunteers forming a placebo group were provided a dosage of 4 placebo capsules (containing corn starch) per day for 15 days, in accordance with the same administration protocol (i.e., 2 placebo capsules in the morning before breakfast, and 2 placebo capsules in the evening before bedtime).

On day 15, blood cytokine levels of the individuals within the test subject group and the placebo group were tested using flow cytometry (FlowCytomix™). Levels of IL-2, TNF-α, and IFN-γ indicative of the amount of Tₕ-1 cells; IL-1β, IL-4, IL-6, and IL-5 indicative of the level of Tₕ-2 cells; and IL-17/IL-22 and IL-9/IL-10 indicative of the amount of Tₕ-17 and T_{reg} cells, respectively, were tested.

Results

FIG. 3 shows an effect of the second anti-autoimmune-disorder composition according to present disclosure on the level of Tₕ-1, Tₕ-2, Tₕ-17, and T_{reg} cells. Placebo group results were normalized to 100. As a result of the administration of the second anti-autoimmune-disorder composition, the level of Tₕ-1 cells in the test subject group significantly decreased as compared to the placebo group, particularly as evidenced by a very significant or dramatic decrease in the level of TNF-α (e.g., a decrease of approximately 65%), and a very significant decrease in the level of IFN-γ (e.g., a decrease of more than 35%, or about 40%). The level of Tₕ-2 cells in the test subject group also significantly decreased as compared to the placebo group, particularly as evidenced by a significant or very significant decrease in the levels of IL-4 (which exhibited a decrease of approximately 35%) and IL-6 (which exhibited a decrease of more than 40%, or about 45%-50%). As further illustrated by FIG. 3, the level of Tₕ-17 very significantly decreased in test subject group compared to the placebo group, as evidenced by a very significant drop (e.g., by approximately 50%) in IL-17 level. Additionally, the level of T_{reg} cells in the test subject group significantly decreased in the test subject group as compared to the placebo group, as indicated by significant or notable decreases in both IL-9 and IL-10 (each of which exhibited decreases of approximately 20%-25%).

Conclusion

Experiments in example 3 indicated that the second anti-autoimmune-disorder composition can support or facilitate the modulation of immune cell levels, specifically by significantly decreasing the levels of Tₕ-1, Tₕ-2, Tₕ-17, and T_{reg} cells. These results support the second anti-autoimmune-disorder composition's ability to facilitate or provide anti-autoimmune-disorder immunomodulation effects, particularly with respect to dramatically decreasing levels of TNF-α, significantly or very significantly decreasing the level of IFN-γ, significantly or very significantly decreasing the levels of IL-4 and IL-6, very significantly decreasing the level of IL-17, and significantly decreasing the levels of IL-9 and IL-10, each of which can play a role in autoimmune disorders involving metabolic dysfunction, for instance, diabetes. Consequently, the second anti-autoimmune-disorder composition appears well suited for supporting, facilitating, or providing at least anti-diabetes immunomodulation.

Representative Aspects of Antipode Immunomodulation Protocols

As indicated above, an anti-pathogen composition in accordance with an embodiment of the present disclosure can increase (e.g., significantly or dramatically) one or more Tₕ cell/cytokine levels; and an anti-autoimmune-disorder composition can decrease (e.g., significantly or dramatically) one or more Tₕ cell/cytokine levels. Thus, an anti-pathogen composition and an anti-autoimmune-disorder composition in accordance with an embodiment of the present disclosure can provide antipode immunomodulatory effects relative to each other.

For providing antipode immunomodulation, the anti-pathogen composition and the anti-autoimmune-disorder composition can be delivered or administered to the subject or subject tissues at separate, segregated, or distinct times or time intervals to thereby allow separate or segregated absorption thereof by the subject, and the generation of antipode anti-pathogen and anti-autoimmune-disorder immunomodulatory effects within the subject in a manner that is based upon or which corresponds to such separate or segregated absorption. Depending upon embodiment details, the anti-pathogen composition and the anti-autoimmune-disorder composition can be provided as separate formulations/delivery vehicles, or a single time-release formulation/delivery vehicle. One or both of the anti-pathogen composition and the anti-autoimmune-disorder composition can exist in a previously described form (e.g., capsule, powder, liquid, etc . . . ).

In accordance with particular embodiments of the present disclosure, the separate or segregated delivery of an anti-pathogen composition and an anti-autoimmune-disorder composition to the subject's body in predetermined amounts at distinct or predetermined times can occur in accordance with an antipode immunomodulation protocol or regime that is expected to synergistically support, enhance, and/or improve the health or immune profile of the subject by way of facilitating or driving two separate, distinct, or distinguishable characteristic types of antipode or generally antipode immune system responses within the subject's body, namely, an anti-pathogen response and an anti-autoimmune-disorder response, in a manner that is intended to ensure that neither of these two characteristic types of immune system responses occurs in isolation on a long-term basis in the absence of the other of these two characteristic types of immune system responses. Consequently, neither of these two characteristic types of immune system responses is expected to dominate (e.g., pathologically dominate) the subject's overall immune response profile over the other characteristic type of immune system response on a long-term basis, thereby reducing, greatly reducing, or minimizing the likelihood that a chronic immune system imbalance associated with either one of these two characteristic types of immune system responses will occur or be exacerbated within the subject's body. An antipode immunomodulation protocol or regime in accordance with embodiments of the present disclosure is intended to synergistically modulate the subject's immune system in an intentionally fluctuating yet appropriately proportioned or balanced manner that facilitates and/or provides both anti-pathogen effects and anti-autoimmune-disorder effects within the subject's body, yet which avoids over-stimulating as well as over-suppressing the subject's immune system responses. Aspects of an antipode immunomodulation protocol in accordance with embodiments of the present disclosure can be predetermined, or established or tailored for purpose of facilitating the restoration and/or maintenance of healthy immune system function in subjects whose immune systems are compromised, and/or exhibit, have exhibited, or have an increased likelihood of exhibiting immune system dysfunction or dysregulation, such as an autoimmune disorder.

Embodiments in accordance with the present disclosure can modulate a subject's immune system in a manner that (a) facilitates in subjects experiencing symptoms associated with pathogen attack, subjects frequently exposed to pathogens, or subjects who have cancer the reduction, eradication, or elimination of pathogens and/or cancer cells from the subject's body, and which avoids inducing or establishing an autoimmune disorder type of response within the subject as a result of over-stimulation of the subject's immune system; and which (b) facilitates in subjects who have an autoimmune disorder or autoimmune dysfunction/dysregulation at least a reduction in autoimmune disorder symptoms, and which also reduces a likelihood of compromising the subject's immune response to pathogens and/or cancer cells as a result of over-suppressing the subject's immune system.

As one representative example, for a subject with cancer or cancer symptoms who also exhibits symptoms of an autoimmune or autoimmune-like disorder, dysregulation, or dysfunction (e.g., chronic fatigue syndrome, diabetes, multiple sclerosis, or Parkinson's disease), an anti-pathogen/anti-cancer composition in accordance with an embodiment of the present disclosure can be administered to the subject one or more times per day, and an anti-autoimmune-disorder composition in accordance with an embodiment of the present disclosure can separately be administered to the subject one or more times per day. For example, an anti-pathogen/anti-cancer composition can be administered to the subject in an alternating manner relative to an anti-autoimmune-disorder composition, where the administration of each type of composition is separated or segregated in time by at least approximately 2-6 hours. The pathogen/anti-cancer composition can be administered to the subject, for instance, once in the morning before breakfast and once at night before bedtime; and the anti-autoimmune-disorder composition can be administered to the subject one or more times during the day.

As another representative example, for a subject who exhibits symptoms of an autoimmune or autoimmune-like disorder, dysregulation, or dysfunction and who is exposed to or is experiencing symptoms of acute viral attack (e.g., associated with a flu virus), an anti-pathogen composition in accordance with an embodiment of the present disclosure can be administered to the subject once in the morning before breakfast and once at night before bedtime, and an anti-autoimmune-disorder composition can be administered to the subject once at mid-morning (e.g., approximately 10:30 A.M.) and once at mid-afternoon or late afternoon (e.g., approximately 4:00 P.M.).

As a further representative example, for a subject who has tested positive for the human immunodeficiency virus (HIV) who also exhibits symptoms of an autoimmune or autoimmune-like disorder, dysregulation, or dysfunction, predetermined amounts of an anti-pathogen composition in accordance with an embodiment of the present disclosure can be administered to the subject one or more times per day (e.g., once in the morning before breakfast and once at night before bedtime, and possibly one or more times during the day such as before lunchtime, mid-afternoon, or early evening) based upon biological markers associated with the progression of HIV infection, and an anti-autoimmune-disorder composition in accordance with an embodiment of the present disclosure can be administered to the subject at one or more times during the day, where administration of the anti-autoimmune-disorder composition is separated or segregated from administration of the anti-pathogen composition by approximately 2-6 hours.

Aspects of particular embodiments of the present disclosure address at least one aspect, problem, limitation, and/or disadvantage associated with existing compositions capable of providing immunomodulatory effects. While features, aspects, and/or advantages associated with certain embodiments have been described in the disclosure, other embodiments may also exhibit such features, aspects, and/or advantages, and not all embodiments need necessarily exhibit such features, aspects, and/or advantages to fall within the scope of the disclosure. It will be appreciated by a person of ordinary skill in the art that several of the above-disclosed systems, components, processes, or alternatives thereof, may be desirably combined into other different systems, components, processes, and/or applications. In addition, various modifications, alterations, and/or improvements may be made to various embodiments that are disclosed by a person of ordinary skill in the art within the scope of the present disclosure.

1. A photychemical composition for facilitating or providing immunomodulatory effects in a subject, the composition comprising:
an extract of mangosteen providing both alpha mangostin and gamma mangostin;
an extract of defatted soybean;
an extract of gotu kola; and
an extract of black sesame,
wherein an amount of the extract of black sesame is less than or equal to 45% of the amount of an amount of a combination of the extract of mangosteen, the extract of defatted soybean, and the extract of gotu kola such that the immunomodulatory effects facilitated or provided by the composition are predominantly anti-pathogen/anti-cancer immunomodulatory effects.

2. The composition of claim 1, wherein the anti-pathogen/anti-cancer immunomodulatory effects are associated with increases in at least some of Th-1 cells, Th-17 cells, and Treg cells in the subject.

3. The composition of claim 1, wherein the anti-pathogen/anti-cancer immunomodulatory effects are associated with increases in at least some of IL-2, TNF-α, IFN-γ, IL1β, IL-17, IL-22, and IL-9 in the subject.

4. The composition of claim 1, wherein an increase in at least one of IL-17 and IL-22 is greater than approximately 400%.

5. The composition of claim 1, wherein an increase in each of IL-17 and IL-22 is greater than approximately 400%.

6. A phytochemical composition for facilitating or providing anti-autoimmune-disorder immunomodulatory effects in a subject, the composition comprising:
an extract of mangosteen providing both alpha mangostin and gamma mangostin;
an extract of defatted soybean; and
an extract of defatted black sesame.

7. The composition of claim 6, wherein the anti-autoimmune-disorder immunomodulatory effects are associated with decreases in at least some of Th-1 cells, Th-2 cells, Th-17 cells, and Treg cells.

8. The composition of claim 6, wherein the anti-autoimmune-disorder immunomodulatory effects are associated with decreases in at least some of IL-2, TNF-α, IFN-γ, IL-4, IL-6, IL-5, IL-17, IL-9, and IL-10.

9. The composition of claim 8, wherein a reduction of TNF-α is approximately 90%.

10. The composition of claim 8, wherein a reduction of IL-17 is greater than approximately 40%.

11. The composition of claim 5, wherein the composition includes an extract of gotu kola, and wherein an amount of the extract of gotu kola is less than or equal to 45% of the amount of a combination of the extract of mangosteen, the extract of defatted soybean, and the extract of defatted black sesame such that that the immunomodulatory effects provided by the composition are predominantly anti-autoimmune-disorder immunomodulatory effects.

12. The composition of claim 6, wherein the composition includes one of (a) a non-refined extract of gotu kola and a non-refined extract of guava to provide first anti-autoimmune-disorder immunomodulatory effects; and (b) a refined extract of gotu kola and a refined extract of guava to provide second anti-autoimmune-disorder immunomodulatory effects distinguishable from the first anti-autoimmune-disorder immunomodulatory effects.

13. The composition of claim 12, wherein an amount of the non-refined extract of gotu kola or an amount of the refined extract of gotu kola is less than or equal to 45% of the amount of a combination of the extract of mangosteen, the extract of defatted soybean, and the extract defatted black sesame such that that the immunomodulatory effects provided by the composition are predominantly anti-autoimmune-disorder immunomodulatory effects.

14. (canceled)

15. (canceled)

16. A method for supporting or providing fluctuating modulation of immune function to a subject, the fluctuating modulation of immune function including anti-pathogen/anti-cancer immunomodulation and anti-autoimmune-disorder immunomodulation, the method comprising:
administering an anti-pathogen/anti-cancer compositions to the subject; and
administering an anti-autoimmune-disorder compositions to the subject,
wherein the administration of the anti-pathogen/anti-cancer composition is separated from the administration of the anti-autoimmune-disorder composition by at least about 2-6 hours.

17. The method of claim 16, wherein each of the anti-pathogen/anti-cancer composition and the anti-autoimmune-disorder compositions is a phytochemical composition, wherein the anti-pathogen/anti-cancer composition comprises an extract of mangosteen providing both alpha mangostin and gamma mangostin, an extract of defatted soybean, and an extract of gotu kola, and wherein the anti-autoimmune-disorder composition comprises an extract of mangosteen providing both alpha mangostin and gamma mangostin, an extract of defatted soybean, and an extract of defatted black sesame.

18. The method of claim 17, wherein the anti-pathogen/anti-cancer composition further comprises an extract of black sesame in an amount less than or equal to approximately 45% of an amount of a combination of the extract of mangosteen, the extract of defatted soybean, and the extract of gotu kola, and wherein the anti-autoimmune-disorder composition further comprises an extract of gotu kola in an amount less than or equal to approximately 45% of an amount of a combination of the extract of mangosteen, the extract of defatted soybean, and the extract of defatted black sesame.

19. (canceled)
